(19) United States
(12) Patent Application Publication
(10) Pub. No.: US 2007/0161986 A1
(43) Pub. Date: Jul. 12, 2007

(54) Polyaxial fastener assembly

(76) Inventor: Mark M. Levy, Raanana (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/299,817

(22) Filed: Dec. 13, 2005

(51) Int. Cl. A61F 2/30

(52) U.S. Cl. 606/61

(57) ABSTRACT

A polyaxial fastener assembly including a mechanical fastener having a proximal end and a distal end, the proximal end including a contact stress element, an articulating element disposed on the mechanical fastener adjacent and distal to the contact stress element, the articulating element having a convex surface and the contact stress element having a convex surface, one of the convex surfaces facing the proximal end and the other convex surface facing the distal end, and a receiver member adapted to receive therein the mechanical fastener, the receiving member having a first aperture having a concave bearing surface that articulates with the convex surface of the articulating element, a second aperture adapted for receiving therein an auxiliary mechanical fastener, and a third aperture for receiving therein a connector element.

### FIELD OF THE INVENTION

This invention relates generally to polyaxial fasteners, such as a polyaxial pedicle screw assembly.

### BACKGROUND OF THE INVENTION

Polyaxial pedicle screw assemblies are well known in the art and may be used for connecting vertebrae or other spinal structure to rods in spinal surgery. For example, U.S. Pat. No. 5,443,467 to Biederman incorporates a ball joint at the connection to the rod to allow the surgeon some flexibility in placing the screws. Tightening a nut on the screw compresses the ball joint components to lock the angular position of the ball joint.

U.S. Pat. No. 6,869,433 to Glascott describes a polyaxial pedicle screw assembly that incorporates a concave portion on a receiver which mates with a convex surface on a head of the screw to form a ball joint. The radius of at least a portion of the concave surface is less than a radius of the mating convex portion whereby to create an interference fit.

### SUMMARY OF THE INVENTION

The present invention seeks to provide an improved polyaxial pedicle fastener assembly, as is described more in detail hereinbelow. The present invention may be used to fasten different elements to spinal structure. For example, the invention may be used to fasten fusion rods between adjoining vertebrae. Alternatively, the invention may be used to fasten the left and right sides pedicles of the same vertebra together. The invention may also be used to connected bones or sections of the same bone in other parts of the body.

There is thus provided in accordance with an embodiment of the invention a polyaxial fastener assembly including a mechanical fastener having a proximal end and a distal end, the proximal end including a contact stress element, an articulating element disposed on the mechanical fastener adjacent and distal to the contact stress element, the articulating element having a convex surface and the contact stress element having a convex surface, one of the convex surfaces facing the proximal end and the other convex surface facing the distal end, and a receiver member adapted to receive therein the mechanical fastener, the receiving member having a first aperture having a concave bearing surface that articulates with the convex surface of the articulating element, a second aperture adapted for receiving therein-an auxiliary mechanical fastener, and a third aperture for receiving therein a connector element, the third aperture being arranged with respect to the first and second apertures such that a connector element disposed in the third aperture is disposed between the contact stress element and the auxiliary mechanical fastener, wherein tightening of the auxiliary mechanical fastener presses the connector element against the contact stress element. For example, the mechanical fastener may be a screw, such as a pedicle screw with a threaded shaft. The mechanical fastener may be coated for different proposes, with antibiotics, enzymes, growth factors or cytokines to encourage bone growth and differentiation, ceramics or hydroxi-appatite for better bone attachment by enhancing bone integration, or may be used in conjunction with cells (e.g., from bone graft, cell culture expansion or genetically engineered sources), bone substitutes or bone cement of any kind, or any combination thereof.

The convex surface of the articulating element may face the distal end and the convex surface of the contact stress element may face the proximal end.

The convex surface of the articulating element and the convex surface of the contact stress element may share a common longitudinal axis of symmetry. In addition, the mechanical fastener has a shank with a longitudinal axis that may be identical to the common longitudinal axis of symmetry.

In accordance with an embodiment of the invention the convex surface of the articulating element may have a radius of curvature greater than a radius of curvature of the convex surface of the contact stress element. The convex surfaces of the articulating element and the contact stress element may be hemi-spherical.

### BRIEF DESCRIPTION OF THE DRAWINGS

The present invention will be understood and appreciated more fully from the following detailed description taken in conjunction with the drawings in which:

FIGS. 1A and 1B are simplified pictorial and sectional illustrations, respectively, of a polyaxial fastener assembly, constructed and operative in accordance with an embodiment of the present invention, showing a mechanical fastener, a receiver member and a connector element;

FIG. 2 is a close-up illustration of the receiver member, constructed and operative in accordance with an embodiment of the present invention; and

FIG. 3 is a sectional illustration of the polyaxial fastener assembly.

### DETAILED DESCRIPTION OF EMBODIMENTS

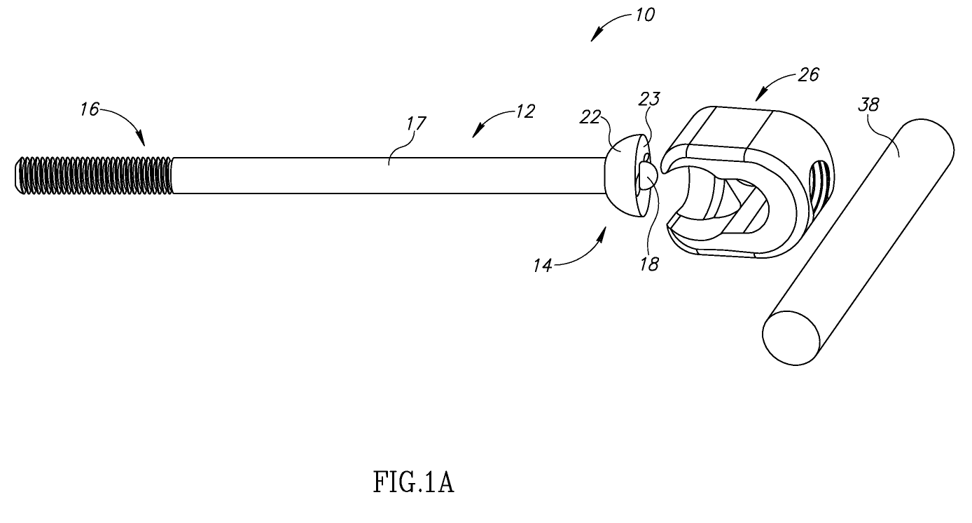

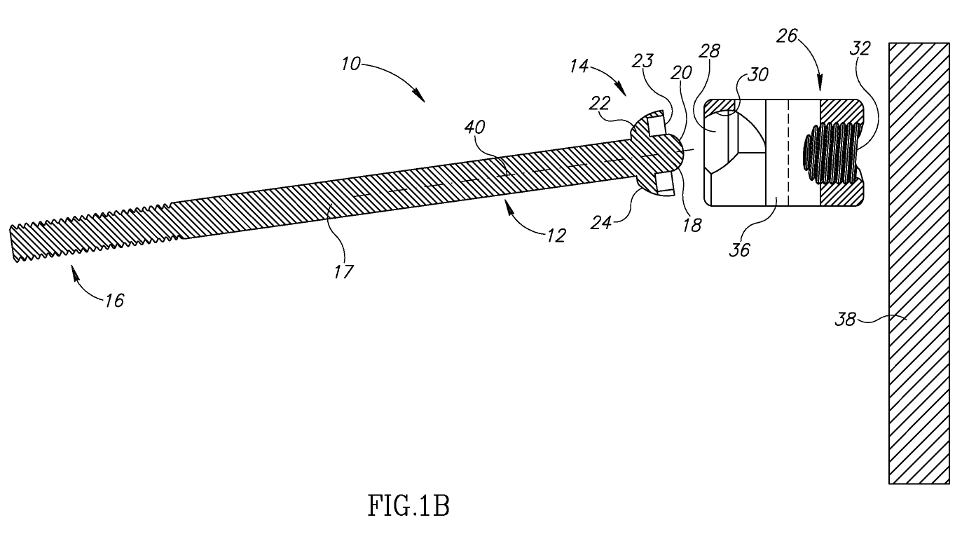

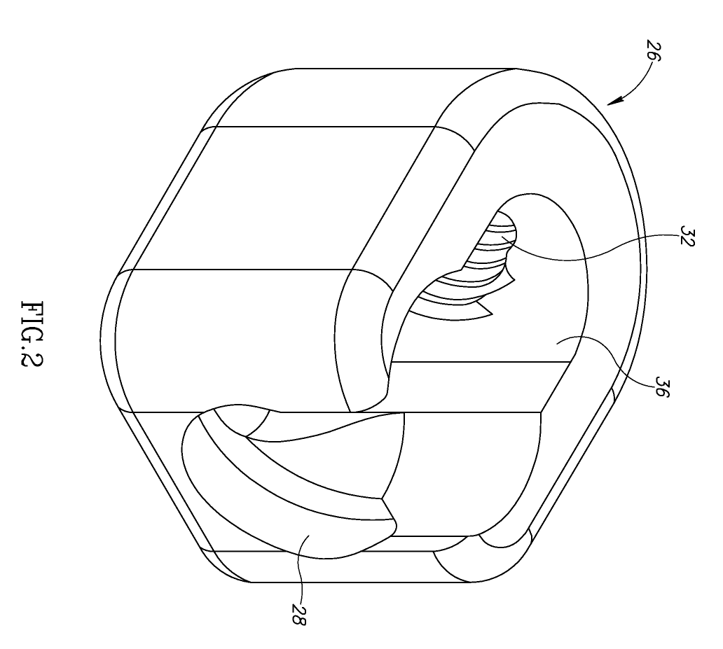

Reference is now made to FIGS. 1A and 1B, which illustrate a polyaxial fastener assembly 10, constructed and operative in accordance with an embodiment of the present invention.

In a non-limiting embodiment of the invention, the polyaxial fastener assembly 10 includes a mechanical fastener 12 having a proximal end 14 and a distal end 16. The mechanical fastener 12 may have a shaft 17 with the distal end 16 being threaded to form a screw (for example, without limitation, a pedicle screw, a pedicle anchor device, a blocking screw for a pedicle anchoring device or any other device for attachment to the pedicle).

The proximal end 14 may include a contact stress element 18 that has a convex or a conical (or any combination thereof) surface 20. An articulating element 22 may be disposed on the mechanical fastener 12 adjacent and distal to the contact stress element 18. The articulating element 22 may have a convex surface 24. The convex surfaces 20 and 24 may face in opposite directions. In the illustrated embodiment, the convex surface 24 of the articulating element 22 may face the distal end 16, whereas the convex surface 20 of the contact stress element 18 may face the proximal end 14. The phrase “convex surface facing a direction” means the outwardly curved surface points in that direction.

The portion of articulating element 22 that faces proximally may be formed with a screwdriver interface element 23 (e.g., one or more indentations or protrusions) for a screwdriver or screwdriver-like device, in order to enable tightening the mechanical fastener 12.

The mechanical fastener 12 with its contact stress element 18 and articulating element 22 may be constructed of any suitable, rigid, medically safe material, such as but not limited to, stainless steel alloy (e.g., AISI 316L), titanium or titanium alloy or chrome cobalt alloy, PEEK, shape memory alloys or polymers, but also from any other natural, synthetic, resorbable or combination of materials safe for use in bone.

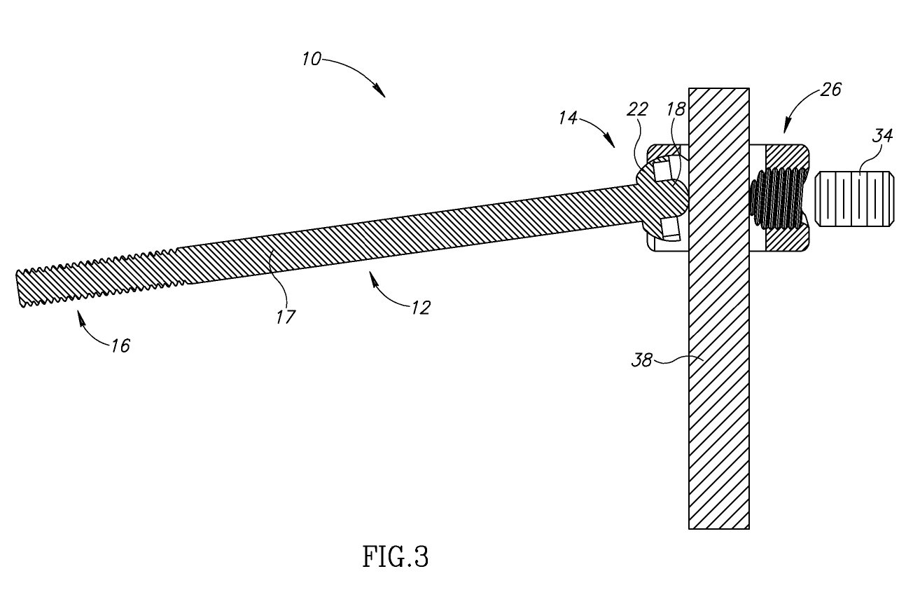

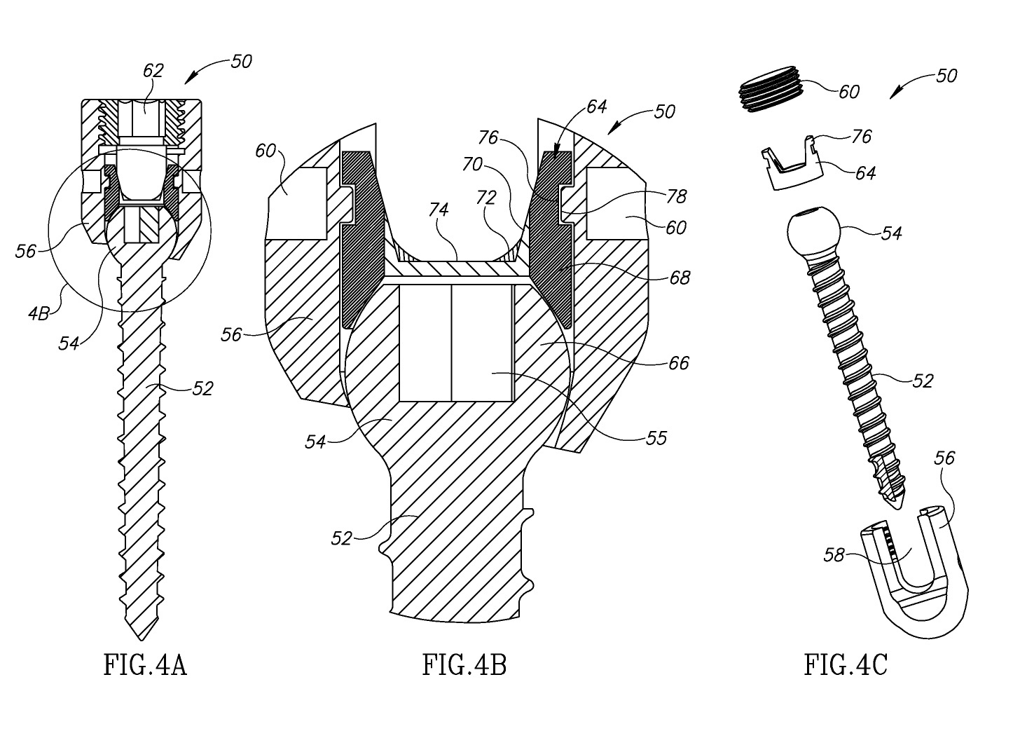

A receiver member 26 may receive therein the mechanical fastener 12. The receiving member 26 may have a first aperture 28 having a concave bearing surface 30 that articulates with the convex surface 24 of the articulating element 22. A second aperture 32 may be formed in receiver member 26 for receiving therein an auxiliary mechanical fastener 34 (shown in FIG. 3, e.g., a set screw, in which case the second aperture 32 is suitably tapped). A third aperture 36 (e.g., a through hole or a blind hole) may be formed in receiver member 26 for receiving therein a connector element 38. The third aperture 36 is arranged with respect to the first and second apertures 28 and 32 such that connector element 38 is disposed between the contact stress element 18 and the auxiliary mechanical fastener 34, wherein tightening of the auxiliary mechanical fastener 34 presses the connector element 38 against the contact stress element 18. The first and second apertures 28 and 32 may be collinear and the third aperture 36 may be perpendicular to them.

The connector element 38 is illustrated as a rod with a cylindrical cross section. However, the invention is not limited to this construction, and connector element 38 may be any slender elongate element, such as but not limited to, a bar of hexagonal, rectangular or square cross section, a rod of elliptical cross section, and many others. The receiver member 26 and the connector element 38 may be constructed of the same or different material as the mechanical fastener 12.

The convex surface 24 of the articulating element 22 and the convex surface 20 of the contact stress element 18 may share a common longitudinal axis of symmetry 40, which may be identical to the longitudinal axis of shank 17. In other words, the radii of curvature of the convex surfaces 24 and 20 of the articulating element 22 and the contact stress element 18, respectively, both lie on the common longitudinal axis of symmetry 40. Moreover, the radii of curvature of the convex surfaces 24 and 20 of the articulating element 22 and the contact stress element 18, respectively, may share a common center. In this manner, the connector element 38 will always be fastened by auxiliary mechanical fastener 34 against the contact stress element 18 and the convex surface 24 of the articulating element 22 will always contact the concave bearing surface 30, no matter what angle connector element 38 is oriented in space relative to the mechanical fastener 12. Alternatively, the radii of curvature of the convex surfaces 24 and 20 of the articulating element 22 and the contact stress element 18, respectively, may have different centers.

It is noted that prior art polyaxial screws can still swivel after a rod or other element is tightened against the polyaxial head. This means that in the prior art, no moment is transferred through the screw, but rather only a force. In the present invention, if the convex surfaces 24 and 20 of articulating element 22 and contact stress element 18 share a common center, both moments and forces are transmitted through the screw. Alternatively, the articulating element 22 and the contact stress element 18 may be configured so that no moment is transferred through the screw as in prior art polyaxial screws.

In accordance with an embodiment of the invention the convex surface 24 of the articulating element 22 may have a radius of curvature greater than a radius of curvature of the convex surface 20 of the contact stress element 18. The convex surfaces 24 and 20 of the articulating element 22 and the contact stress element 18, respectively, may be hemi-spherical. The concave bearing surface 30 may also be accordingly hemi-spherical. However, the invention is not limited to this shape, and encompasses without limitation, ellipsoidal, conical and polygonal shapes of all kinds, preferably chosen in accordance with the shape of connector element 38. (It is noted that in mathematics, the term “convex” also applies to polygons and is defined as a contour containing no interior angle greater than 180°. Similarly, the term “concave” also applies to polygons and is defined as a contour containing an interior angle greater than 180°.)

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A polyaxial fastener assembly comprising:
a mechanical fastener having a proximal end and a distal end, the proximal end comprising a contact stress element;
an articulating element disposed on said mechanical fastener adjacent and distal to said contact stress element, said articulating element having a convex surface and said contact stress element having a convex surface, one of said convex surfaces facing the proximal end and the other convex surface facing the distal end; and
a receiver member adapted to receive therein said mechanical fastener, said receiving member having a first aperture having a concave bearing surface that articulates with said convex surface of said articulating element, a second aperture adapted for receiving therein an auxiliary mechanical fastener, and a third aperture for receiving therein a connector element, said third aperture being arranged with respect to said first and second apertures such that a connector element disposed in said third aperture is disposed between said contact stress element and said auxiliary mechanical fastener, wherein tightening of said auxiliary mechanical fastener presses the connector element against said contact stress element.

2. The polyaxial fastener assembly according to claim 1, wherein said mechanical fastener comprises a screw with a threaded shaft.

3. The polyaxial fastener assembly according to claim 1, wherein said convex surface of said articulating element faces the distal end and said convex surface-of said contact stress element faces the proximal end.

4. The polyaxial fastener assembly according to claim 1, wherein said convex surface of said articulating element and said convex surface of said contact stress element share a common longitudinal axis of symmetry.

5. The polyaxial fastener assembly according to claim 4, wherein radii of curvature of said convex surfaces of said articulating element and said contact stress element share a common center.

6. The polyaxial fastener assembly according to claim 4, wherein said mechanical fastener has a shank with a longitudinal axis identical to said common longitudinal axis of symmetry.

7. The polyaxial fastener assembly according to claim 1, wherein said convex surface of said articulating element has a radius of curvature greater than a radius of curvature of said convex surface of said contact stress element.

8. The polyaxial fastener assembly according to claim 1, wherein said convex surface of said articulating element is hemi-spherical.

9. The polyaxial fastener assembly according to claim 1, wherein said convex surface of said contact stress element is at least one of a hemi-spherical surface and a conical surface.

10. The polyaxial fastener assembly according to claim 1, further comprising a connector element disposed in said third aperture and fixed between said contact stress element and said auxiliary mechanical fastener.

11. The polyaxial fastener assembly according to claim 1, wherein said first and second apertures are aligned on a common axis and said third aperture is generally perpendicular to said common axis.

12. The polyaxial fastener assembly according to claim 1, wherein a portion of said articulating element that faces proximally is formed with a screwdriver interface element.

13. A polyaxial fastener assembly comprising:
a mechanical fastener having a proximal end and a threaded distal end, the proximal end comprising a contact stress element;
an articulating element disposed on said mechanical fastener adjacent and distal to said contact stress element, said articulating element having a convex surface that faces the distal end and said contact stress element having a convex surface that faces the proximal end, wherein said convex surface of said articulating element and said convex surface of said contact stress element share a common longitudinal axis of symmetry, wherein said convex surface of said articulating element has a radius of curvature greater than a radius of curvature of said convex surface of said contact stress element; and
a receiver member adapted to receive therein said mechanical fastener, said receiving member having a first aperture having a concave bearing surface that articulates with said convex surface of said articulating element, a second aperture adapted for receiving therein an auxiliary mechanical fastener, and a third aperture for receiving therein a connector element, said third aperture being arranged with respect to said first and second apertures such that a connector element disposed in said third aperture is disposed between said contact stress element and said auxiliary mechanical fastener, wherein tightening of said auxiliary mechanical fastener presses the connector element against said contact stress element, and wherein said first and second apertures are aligned on a common axis and said third aperture is generally perpendicular to said common axis.

14. The polyaxial fastener assembly according to claim 13, wherein a portion of said articulating element that faces proximally is formed with a screwdriver interface element.

15. The polyaxial fastener assembly according to claim 13, wherein said mechanical fastener has a shank with a longitudinal axis identical to said common longitudinal axis of symmetry.

16. The polyaxial fastener assembly according to claim 13, wherein said convex surface of said contact stress element is conical.
